(19) United States
(12) Patent Application Publication
(10) Pub. No.: US 2014/0296707 A1
(43) Pub. Date: Oct. 2, 2014

(54) LUMINESCENT COMPOSITION AS BIOMARKER IN A BIRD'S EGG, CORRESPONDENCE DEVICE AND METHOD

(71) Applicants: Marc Massonneau, Tilliers sur Avre (FR); Laurent Mogenet, Libourne (FR)

(72) Inventors: Marc Massonneau, Tilliers sur Avre (FR); Laurent Mogenet, Libourne (FR)

(21) Appl. No.: 13/995,636

(22) PCT Filed: Dec. 22, 2011
(86) PCT No.: PCT/EP2011/073869

§ 371 Date: Jul. 22, 2013

(30) Foreign Application Priority Data
Dec. 27, 2010 (FR) 1061278

(51) Int. Cl. A61K 49/00; A61B 5/00; A61D 7/00

(52) U.S. Cl. CPC A61K 49/0034; A61D 7/00; A61B 5/0071; A61B 5/4848
USPC 600/431; 548/427

(57) ABSTRACT

Luminescent composition injectable into a bird's egg characterized in that it comprises a luminescent compound forming a biomarker for a product having a vaccinal, therapeutic or diagnostic activity

### TECHNICAL FIELD OF THE INVENTION

The invention relates to the use of compounds capable of emitting a luminescent signal that can be detected in a bird egg.

The invention relates specifically to the use of these biomarkers in a method for detecting and/or quantifying a biological phenomenon for vaccine and/or therapeutic and/or diagnostic purposes in a bird egg.

The invention more specifically is intended for the field of in ovo vaccination.

### TECHNOLOGICAL BACKGROUND

Optical imaging is increasing in popularity in the biomedical field, with numerous preclinical and human applications in fields such as cancer, for the detection of tumours, or neuroscience, for brain imaging.

Thus, as was described in the patent document published under number WO 2008/025006 on mice and small animals, photons emitted by the dispersion of cells marked by a luminescent compound excited by light spread through the tissue of these mammals. Even if many of these diffused photons are absorbed, a fraction reaches the surface where they can be detected by a camera that records the spatial distribution of the photons emitted by the surface in two dimensions (2D).

However, even in the case of a single fluorescent probe in a mouse, tissue autofluorescence makes it difficult to obtain a good resolution.

To attempt to overcome these deficiencies, two main areas have been explored:
adaptation of optical imaging devices according to the orientations described in the patent document published under number WO2007/144542;
adaptation of luminescent probes: numerous probes have been developed, but the applicant has focused more specifically on biomarkers, such as those described in the patent document published under number WO2006/129036. However, this information does not enable and does not suggest applying optical imaging to eggs.

### OBJECTIVES OF THE INVENTION

One of the preferred applications of the invention is applied to in ovo vaccination.

A certain number of techniques have been developed until now to enable in ovo vaccination of embryos, that is, vaccination of embryos when they are still in the egg. It is indeed recognized that these in ovo vaccinations make it possible to reduce costs, significantly automate the vaccination, reduce stress and increase the success rate of the vaccination of chicks after hatching.

One of the difficulties of this in ovo vaccination operation lies in the testing of the vaccination, as the grade of the egg may vary significantly. Indeed, a fertilized egg includes a plurality of distinct compartments, including the outer shell, the air chamber, the allantoic fluid, the amniotic fluid and the embryo. Vaccination is effective only if the product is injected into the embryo or the amniotic fluid.

Practically speaking, it is difficult to ensure that the injection has been performed successfully, in particular on automated bird egg processing lines, such as processing plants for eggs of future meat chickens.

One of the solutions commonly used these days consists of mixing the injected product with a dye and sampling a large number of eggs on the vaccination line to verify the quality of the injection. If the egg thus tested is considered to be noncompliant, then the injection device is re-sampled.

One of the disadvantages of this method is that it requires the destruction of some hundreds and even thousands of eggs, therefore embryos, without nevertheless ensuring good reproducibility of the vaccination, and is costly in terms of personnel and time. Another disadvantage associated with this massive destruction of eggs is the impossibility of applying this method routinely, and the invasive nature, which is incompatible with the standards of automation, productivity and well-being.

There is also a need for a testing method in order to be able to ensure that the vaccination and/or sampling are performed under optimal conditions.

Therefore, the invention is intended to overcome at least some of the disadvantages of the methods and devices of the prior art.

In particular, the invention is intended to provide a non-invasive method for testing the quality of an injection of product into an egg.

The invention is also intended to provide such a method that is reliable not only with one size of egg.

The invention is also intended to propose a device implementing a method according to the invention.

### DESCRIPTION OF THE INVENTION

To do this, the invention relates to any luminescent composition that can be injected into an egg, characterized in that it includes a compound capable of emitting a luminescent (or fluorescent or autoluminescent or chemiluminescent) signal that can be detected in an egg forming a biomarker of a product having a vaccine, therapeutic or diagnostic activity.

It should be noted that the luminescent compounds of the invention used to mark a product having such a vaccinal, therapeutic or diagnostic activity clearly differ from the labelled antibodies capable of being detected by excitation light, known from the application WO2010/103111 and implemented in order to determine in ovo the sex of bird species.

By compound capable of emitting a luminescent signal, we mean any compound that emits radiation as it passes from a stable state to an excited state, or vice versa.

According to a particular embodiment, said luminescent compound is indocyanine green, which will emit photons after the egg has been exposed to an excitation light source.

According to an alternative, the composition also includes a product having a vaccine, therapeutic or diagnostic activity.

The invention also relates to a method for testing an injection of a product for vaccine or therapeutic or diagnostic purposes into an egg, including, in sequence, the following steps:
injection of a composition containing said product and a luminescent compound into an egg;
detection of the signal emitted by the luminescent device through the shell of the egg;
processing of the information collected.

It thus clearly appears that a testing method according to the invention is non-invasive.

In addition, as will become clearer below, the reliability of a method according to the invention is independent of the size of the egg analyzed.

Moreover, it is possible to integrate a method according to the invention in an automatic vaccination line, for example.

The invention therefore lies in the use of a luminescent compound as a marker of the product injected into the egg. The applicant indeed discovered that it is possible to detect the photons emitted by the luminescent compound, in spite of the presence of a mineral barrier, namely the shell of the egg.

According to an advantageous solution, the method includes a step of correlating the signal emitted by the luminescent compound and the location thereof in the egg.

It is possible in this way to obtain a clear and direct indication of the position of the product injected into the egg, and to verify whether this position is suitable or not with respect to the expected effect of the injected product.

According to an advantageous solution, the method includes a step of subjecting the egg to an excitation light source necessary for detection of the signal. The luminescent compound is thus excited by the adapted light source, which can preferably be produced by means of a laser or a suitable light excitation source including, in particular, filters adapted to the wavelengths of the phosphor.

According to an advantageous alternative, said step of subjecting the egg to an excitation light source and said step of detecting the signal are performed on each side of the axis of symmetry of said egg.

Advantageously, said steps of subjection and processing of the information collected are performed for at least two, and preferably at least three, distinct relative angular positions between the egg and said light excitation source.

These distinct angular positions may be obtained while the egg is rotated in place. It is also possible to envisage obtaining these positions by holding the egg still and varying the position of the light emission source and/or the position of the means used to process the collected information.

It is possible in this way to obtain a set of signals representing a three-dimensional “image” of the position of the luminescent compound and therefore that of the injected product, in the egg.

The signals obtained according to the different positions can also be the subject of a selection so as to retain only the best signal in view of said correlation step.

According to an advantageous solution, said signal detection step is performed by means of a digital camera.

Also, according to an alternative of the invention, said step of processing the collected information is performed only on the part of the signal emitted by the upper part of the egg containing the air chamber.

According to a particular embodiment, the method includes a preliminary step of modelling the signal emitted by the luminescent compound when it is located in each of the following areas of the egg:
air chamber;
allantoic fluid;
amniotic fluid;
embryo.

In this case, said analysis step is preferably performed by means of a processing unit wherein the modelled signals obtained in said preliminary step are stored.

In this case, the method advantageously includes a step of comparing the signal emitted by the luminescent compound through the shell of the egg during the analysis step with the modelled signals obtained during said preliminary step.

According to a first approach of the invention, said injection step is performed so as to simultaneously inject said product to be injected and said luminescent compound.

According to a second approach of the invention, said injection step is performed so as to separately inject said product to be injected and said luminescent compound.

In this case, said luminescent compound is capable of binding with the product.

In one or the other case, it is noted that the luminescent compound is intended to occupy a position in the egg coinciding with that of the injected product.

According to a particular embodiment, said luminescent compound is indocyanine green (CAS no. 3599-32-4) with the general formula C₄₃H₄₇N₂NₐO₆S₂:

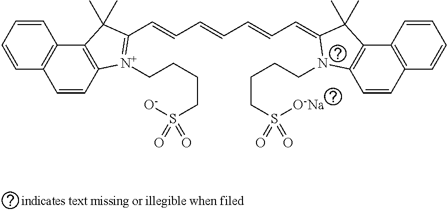

Preferably, this luminescent compound is a compound of general formula of the S-B-A type or derivatives thereof, such as salts, esters or derivatives functionalized with structural elements described and claimed by the patent document published under number FR-2-886 292.

A method according to the invention can be used to test and/or quantify the following in an egg:
a vaccine and/or a pharmaceutical substance;
a diagnostic or prognostic product;
the embryo test:
   the presence or absence of an embryo;
   the qualitative assessment of the embryo.

The invention also relates to any kit characterized in that it includes at least one luminescent composition as described above and in that it also includes an instruction sheet on the procedure for implementing said composition in the context of such a method.

The invention also relates to a device for implementing the steps of subjection and analysis of a method according to the invention, characterized in that it includes:
an area for receiving at least one egg;
optionally at least one light excitation source directed toward said receiving area;
means for detecting the signal emitted by the luminescent compound through the shell of the egg;
means for processing the information collected.

Preferably, said receiving area is rotatably mounted.

The device according to the invention can be in the form of a self-contained device and/or a device integrated in an automated egg processing line, such as an automated vaccination line.

### LIST OF FIGURES

Other objectives, features and advantages of the invention will appear in view of the following description provided solely for non-limiting purposes, and which refers to the appended figures, wherein:

FIG. 1 is a diagrammatic view of an embodiment of a device for implementing a method according to the invention;

FIGS. 2 to 5 are diagrammatic views of different signals emitted by a luminescent compound in a method according to the invention;

FIG. 6 is a set of three images corresponding to digital camera image captures of an egg subjected to an excitation light source (laser) and in the air chamber into which a luminescent compound has been injected;

FIG. 7 is an image corresponding to a digital camera image capture of an egg in the air chamber with which a luminescent compound has been injected and associated with a vertical axis Y on which N=400 horizontal lines are distributed;

FIG. 8 is a graph translating the mean light intensity distribution (Arbitrary Unit of relative intensity) of the image of FIG. 7 according to the vertical axis Y;

FIGS. 9 to 12 show the associated images and the light intensity distribution graphs according to the vertical axis Y, forming modelled signals for eggs injected with the luminescent compound, respectively, into the air chamber, the allantoic fluid, the amniotic fluid and the embryo;

FIG. 13 shows a light intensity distribution graph associated with an egg into which a luminescent compound has been injected;

FIG. 14 shows the same graph as that of FIG. 13, but wherein the whole of the curve thereof corresponds to the light intensity of the part of the egg containing the air chamber (S1) and the whole of the curve thereof corresponding to the light intensity of the rest of the egg (S1) have been identified.

### DETAILED DESCRIPTION OF AN EMBODIMENT OF THE INVENTION

Throughout the remainder of the description, reference is made to the use of a method according to the invention in order to test the injection of a vaccine into an egg.

However, the method according to the invention can be used for other products, for example to test the injection of a therapeutic treatment product or a diagnostic product.

Applied to the testing of a vaccine injection, a method according to the invention includes the following steps:
injecting a composition including said vaccine and a luminescent compound in an egg;
subjecting the egg to a light excitation source;
detecting and analyzing the signal emitted by the luminescent compound through the shell of the egg.

In the context of the embodiment described here, the luminescent compound is the compound of formula C₄₃H₄₇N₂NₐO₆S₂ commonly called indocyanine green (CAS no. 3599-32-4). Another compound called FR99, described and claimed by the patent document published under number FR-2 886 292 was evaluated and results not presented confirm the observations made with indocyanine green. These compounds are chemically inert with respect to the vaccine. The rheological properties of the composition including the luminescent compound are substantially the same as those of the vaccine.

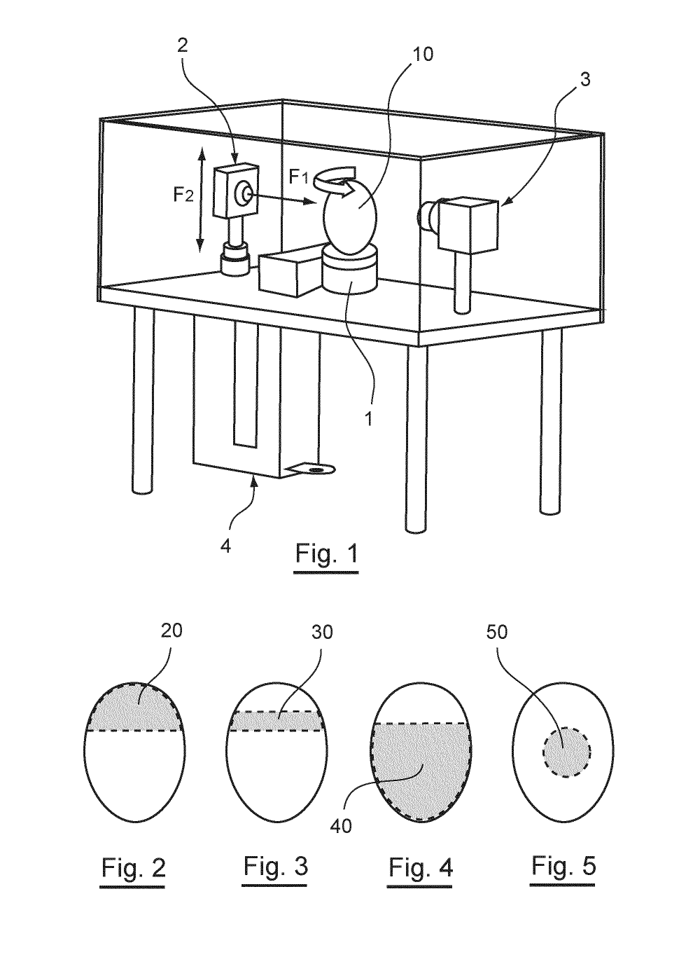

A method according to the invention can, for example, be implemented with a device as shown in FIG. 1.

As shown in this figure, a device for implementing a method for testing a vaccine injection in an egg includes:
an area 1 for receiving an egg 10;
   a laser 2, of which the beam is capable of being directed toward the receiving area, and more specifically toward the egg 10;
   the laser forms a light excitation source of the luminescent compound injected into the egg;
   a digital camera 3, forming means for detecting the signal emitted by the luminescent compound through the shell of the egg;
   a processing unit 4, intended to receive and process the data obtained by the digital camera 3.

As shown in FIG. 1, the receiving area is placed between the laser 2 and the digital camera 3. It is noted that it is not necessary for the laser, the receiving area and the digital camera to be aligned one with respect to another.

According to the present embodiment, the receiving area 1 is rotatably mounted about a vertical axis (as shown by the curved arrow F1), and is coupled to rotational driving means (not shown).

Preliminarily, in the vaccination testing phase using a device as described above, a step is performed of modelling the signal capable of being emitted by the luminescent compound injected into the egg, under light excitation, for each of the following areas of the egg:
the air chamber;
the allantoic fluid;
the amniotic fluid;
the embryo.

Thus, in this preliminary modelling step, the luminescent compound reacts to the light excitation and obtains, through the shell, in return, a signal among those shown in FIGS. 2 to 5.

Thus, when the luminescent compound is located in the air chamber, a luminous area 20 as shown in FIG. 2 is obtained, substantially coinciding with the volume of the air chamber.

When the luminescent compound is located in the allantoic fluid, a luminous area 30 as shown in FIG. 3 is obtained, substantially coinciding with the volume of the allantoic sac.

When the luminescent compound is located in the amniotic fluid, a luminous area 40 as shown in FIG. 4 is obtained, substantially coinciding with the volume occupied by the amniotic fluid.

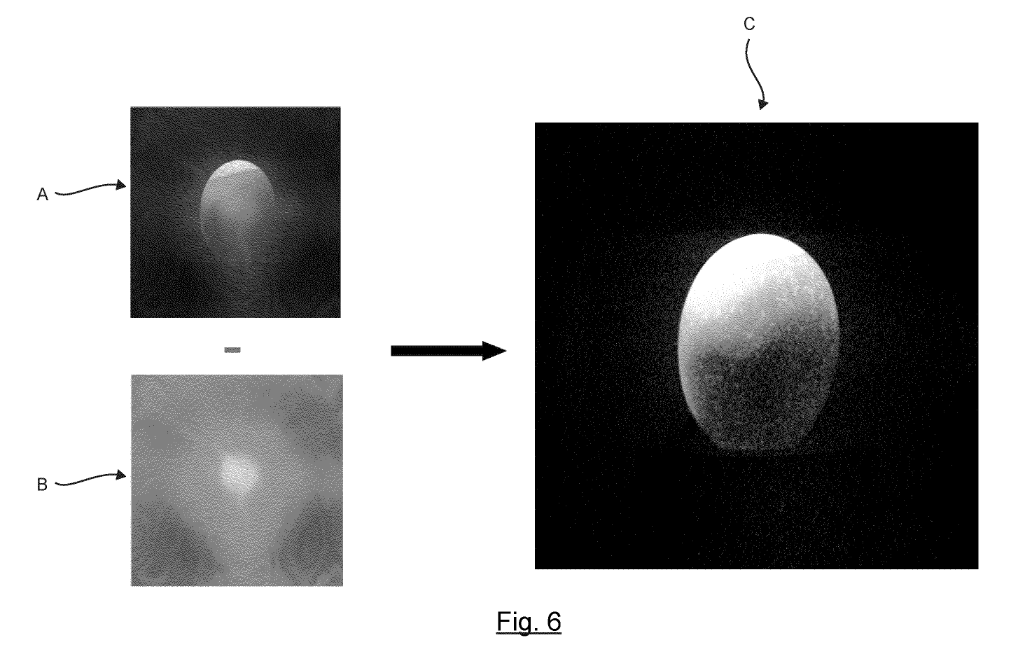

When the luminescent compound is located in the embryo, a luminous area 50 is obtained, as shown in FIG. 5, substantially coinciding with the volume occupied by the embryo (which of course varies according to its stage of development).

These areas 20, 30, 40, 50 are therefore modelled to obtain data in computerized form stored by the processing unit 4.

When an egg is subjected to the testing method according to the invention by means of a device as described above, the vaccine and the luminescent compound are injected into the eggs.

This step can be performed so that the injection of the vaccine and the injection of the luminescent compound are performed simultaneously. It therefore follows that the luminescent compound is located in the same area as the vaccine, as the vaccine and the luminescent compound are injected together, or even mixed.

It is also possible to separately inject the vaccine and the luminescent compound. In this case, the luminescent compound has a chemical composition intended to react with that of the vaccine so that the luminescent compound is placed in the same area of the egg as the vaccine (which may also be the case even if the luminescent compound is injected simultaneously with the vaccine). The chemical compositions of the luminescent compound and the vaccine can be chosen so that the luminescent compound binds to the vaccine.

The eggs to be tested are therefore subjected to the rays of the laser 2, the analysis of the signal emitted by the luminescent compound through the shell of the egg then being performed by the digital camera 3. The image captured by the digital camera 3 is provided in the form of digital data to the processing unit 4, which performs a step of correlating the signal emitted by the luminescent compound and locating it in the egg.

For this, the processing unit performs a step of comparing the signal emitted by the luminescent compound through the shell of the egg, provided by the digital camera, with the modelled signals obtained in the preliminary step having led to the representative stored data, for example, of FIGS. 2 to 5.

Of course, the processing unit is parameterized so as to perform the test in the form of a trial: depending on the product injected, it is expected that the injection of it will be performed in a precise area of the egg and if the data representing the position of the product injected into the egg (this data being obtained by means of the luminescent compound) does not correspond to the data of a modelled position, the test is negative. Of course, if there is a correspondence between the two positions, the result is positive.

To increase the reliability of the method according to the invention, it is possible to subject the egg to light excitation and to analyze the signal emitted by the luminescent compound for at least two angular positions of the egg (around its vertical axis) with respect to the rays of the laser. Preferably, the test is performed when the receiving area 1 is rotated (the receiving area being designed so that the rotation of the receiving area drives that of the egg about its vertical axis).

The method according to the invention was tested so as to demonstrate its relevance in the testing of a vaccine injection. The main results of these different tests performed are presented below in reference to FIGS. 6 to 14.

### Determination of the Optimal Concentration of Luminescent Compound and the Optimal Acquisition Time of the Signal

Twenty-four fertilized eggs at 18.5 days of incubation were subjected to an in ovo injection of an indocyanine green solution at different concentrations, intended to correspond with a vaccine solution marked by a luminescent marker. Different acquisition times of signals captured in image form by the camera 3 were tested.

In all, 720 images were analyzed. This analysis made it possible to demonstrate that:
the optimal concentration of luminescent compound (Cₒₚₜ) is 50 micromoles per litre;
the optimal acquisition time of the signal (Tₒₚₜ) is 90 seconds.

Tests were then performed to model signals emitted by the luminescent compound when it is injected into each of the following areas of an egg:
the air chamber,
the allantoic fluid,
the amniotic fluid,
the embryo.

To this effect, eggs having different fertilization ages, namely: 17, 17.5, 18, 18.5 and 19 days of incubation, were subjected to the injection of indocyanine green at the optimal concentration (Cₒₚₜ) determined above.

For each fertilization age, the injection was performed at different injection depths, namely: in the air chamber of an egg, in the allantoic fluid of a second egg, in the amniotic fluid of a third egg and in the embryo of a fourth egg.

For each of these eggs, three images were captured, before and after injection, by the camera 3 for the optimal acquisition time (Tₒₚₜ) according to three distinct relative positions of the egg with respect to the camera 3 and the laser ray 2. These three distinct positions were obtained by rotating the egg in place about its axis of symmetry of 120°, then again 120°.

The images thus captured by the camera 3 were then processed by the processing unit 4.

This processing first consisted of processing the signal in order to remove the noise generated by the coherent light passing through the egg and not resulting from the presence of indocyanine green. Practically speaking, the signal obtained after injection was subtracted from the signal obtained before injection, and for each egg, for each position and for each injection depth.

FIG. 6 shows the image (A) obtained for an egg in a given position before injection of the luminescent product, the image (B) obtained for the same egg in the same position after injection of the product into its air chamber at the optimal concentration defined above, and, the processed image (C) obtained by processing signals corresponding to images (A) and (B) having consisted of subtracting, from the signal corresponding to image (B), the signal corresponding to image (A).

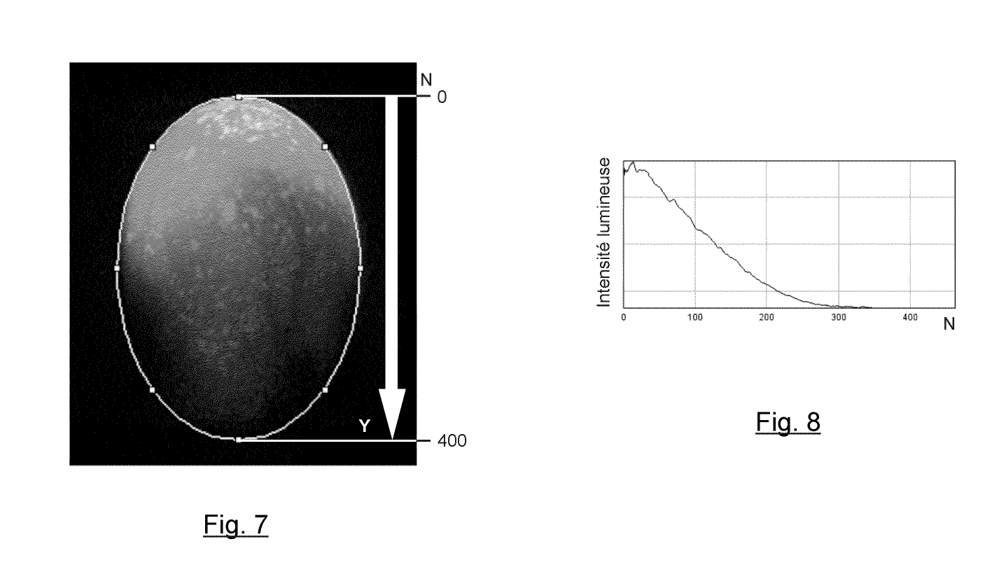

For each egg, the three processed images (C) obtained for the three positions cited above were then analyzed. In reference to FIG. 7, this analysis consisted of performing, for each image, a series of measurements of light intensity and of obtaining an average for each line, from top to bottom of the image according to the Y axis, so as to obtain a distribution of the mean light intensity according to this axis as indicated, for example, in FIG. 8. Practically speaking, 400 measurements were performed for each image (corresponding to 400 horizontal lines of the images), i.e. 1200 measurements per egg.

For each egg, among the three images processed and analyzed, the processed image showing the highest mean light intensity, indicating the maximum presence of fluorescent product, was selected.

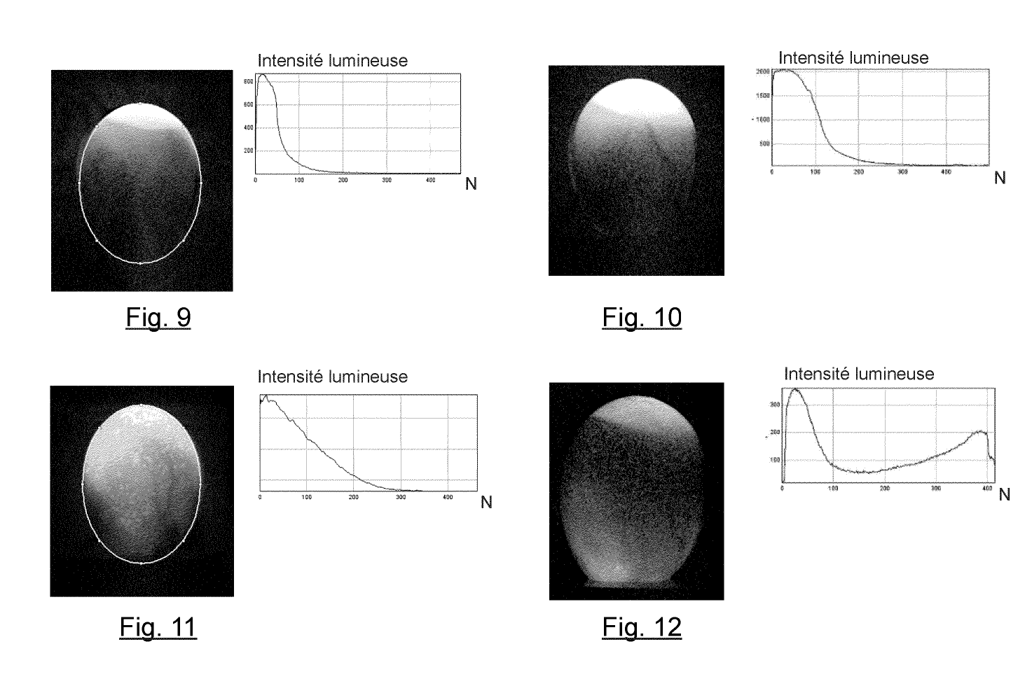

Thus, models of signals emitted by the luminescent compound when it was injected into each of the following areas of the egg: air chamber, allantoic liquid, amniotic liquid, embryo, could be obtained. These modelled signals and the corresponding images are shown in FIGS. 9, 10, 11 and 12, respectively. They each indicate the relative mean light intensity (Arbitrary Unit of relative intensity) (Y-axis) as a function of a number N of lines, in this case 400, indicating the height of the egg according to its vertical axis from its apex (X-axis).

As can be seen in FIGS. 9 to 12, each model shows a very specific profile. In reference to FIG. 13, an overall mean light intensity can therefore be associated with each compartment of the egg. Thus, the signal that will be detected and processed according to the invention for an egg having been subjected to an injection of a product, such as, for example, a vaccine, and luminescent product, may easily be compared to one of these models so as to determine the part of the egg in which the injection actually took place.

On this subject, the shape of these profiles also indicates that it may be possible to envisage confining the comparison step to the part of the signal emitted by the upper part of the egg containing the air chamber, and therefore to process only this part of the signal for said comparison.

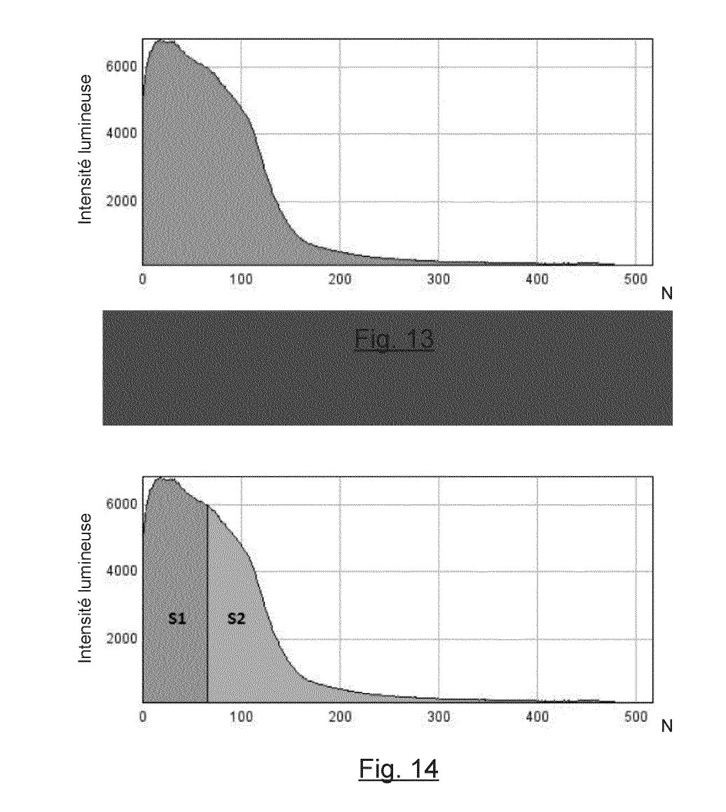

In reference to FIG. 14, it is also noted that each signal can be associated with a ratio S1/S2 indicating the proportion of luminescence emitted by the part of the egg corresponding substantially to that containing the air chamber, with respect to the luminescence emitted by the rest of the egg. Thus, in reference to FIG. 15, an average surface ratio can also be associated with each compartment of the egg. This ratio can also be used to help to determine the part of the egg in which the injection actually took place.

### Correlation

To validate the correlation between the images acquired and the real location of the injected solution, a test was performed on sixty eggs having different fertilization ages, namely 17, 17.5, 18, 18.5 and 19 days of incubation (12 eggs per age).

To do this, 60 embryonated eggs of the indicated ages received a double injection (luminescent compound and blue stain) with locations approximately balanced between the 4 compartments. Then, an optical examination of the egg under the 3 angles, followed by a complete shelling of the egg (enabling the positioning of the blue stain to be characterized), simultaneously generated, for each egg, the data on optical intensity and precise location of the injection.

This test made it possible to confirm that the images acquired by the method according to the invention were closely correlated with the real location of the solution injected into the egg.

1. Luminescent composition capable of being injected into a bird egg, characterized in that it includes a luminescent compound forming a biomarker of a product having a vaccine, therapeutic or diagnostic activity.

2. Composition according to claim 1, characterized in that said luminescent compound is a fluorescent or autoluminescent or chemiluminescent compound.

3. Composition according to claim 1, characterized in that said luminescent compound is indocyanine green or derivatives thereof, such as salts, esters or derivatives functionalized with structural elements.

4. Composition according to claim 1, characterized in that said luminescent compound is of the S-B-A type or derivatives thereof, such as salts, esters or derivatives functionalized with structural elements.

5. Method for testing an embryo in a bird egg, including, in sequence, the following steps:
injection of a composition containing a luminescent compound into a bird egg, the luminescent compound forming a biomarker of a product having a vaccine, therapeutic or diagnostic activity;
detection of the signal emitted by the luminescent device through the shell of the egg;
processing of the information collected.

6. Method for testing an injection of a product for vaccine or therapeutic or diagnostic purposes in an egg, including, in sequence, the following steps:
injection of a composition containing said product and a luminescent compound into an egg;
detection of the signal emitted by the luminescent device through the shell of the egg;
processing of the information collected.

7. Method according to claim 5, characterized in that it includes a step of correlating the signal emitted by the luminescent compound and of locating it in the egg.

8. Method according to claim 5, characterized in that it includes a step of subjecting the egg to a light excitation source necessary for detection of the signal.

9. Method according to claim 8, characterized in that the light excitation source is adapted to the wavelengths of the luminescent compound.

10. Method according to claim 8, characterized in that the excitation light source is a laser or any filter adapted to the wavelengths of the luminescent compound.

11. Method according to claim 5, characterized in that it includes a preliminary step of modelling the signal emitted by the luminescent compound when it is located in each of the following areas of the egg:
air chamber,
allantoic fluid,
amniotic fluid,
embryo.

12. Method according to claim 11, characterized in that said analysis step is performed by means of a processing unit wherein modelled signals obtained in said previous step are stored.

13. Method according to claim 12, characterized in that it includes a step of comparing the signal emitted by the luminescent compound through the shell of the egg during the detection step with the modelled signals obtained in said previous step.

14. Method according to claim 5, characterized in that said injection step is performed so as to simultaneously inject said product to be injected and said luminescent compound.

15. Method according to claim 5, characterized in that said injection step is performed so as to separately inject said product to be injected and said luminescent compound.

16. Method according to claim 14, characterized in that said luminescent compound is capable of binding to the product.

17. Method according to claim 8, characterized in that said step of subjecting the egg to a light excitation source and said step of detecting the signal are performed on each side of the axis of symmetry of said egg.

18. Method according to claim 8, characterized in that said steps of subjection and processing of the information collected are performed for at least two, preferably at least three, distinct relative angular positions between the egg and said light excitation source.

19. Method according to claim 8, characterized in that said step of processing the information collected is performed only on the part of the signal emitted by the upper part of the egg containing the air chamber.

20. Use of a composition according to claim 1 for testing, in an egg, the injection of a vaccine and/or a pharmaceutical substance and/or a diagnostic or prognostic product.

21. Use of a composition according to claim 1 for testing the embryo in an egg.

22. Use of a method according to claim 5 for testing, in an egg, the injection of a vaccine and/or a pharmaceutical substance, and/or a diagnostic or prognostic product.

23. Use of a method according to claim 5 for testing the embryo in an egg.

24. Device for implementing steps of detection and analysis of a method according to claim 5, characterized in that it includes:
an area for receiving at least one egg;
optionally at least one light excitation source directed toward said receiving area;
means for detecting the signal emitted by the luminescent compound through the shell of the egg;
means for processing the information collected.

25. Kit characterized in that it includes at least one composition according to claim 1, and in that it also includes an instruction sheet.
